(19) United States
(12) Patent Application Publication
(10) Pub. No.: US 2011/0005527 A1
(43) Pub. Date: Jan. 13, 2011

(54) ORTHODONTIC APPLIANCES

(75) Inventors: McCance Andrew, Sussex (GB); Caprani Joe, Sussex (GB)

Correspondence Address: STEPTOE & JOHNSON LLP, 2121 AVENUE OF THE STARS, SUITE 2800, LOS ANGELES, CA, 90067, US

(73) Assignee: ORTHO-PRO-TEKNICA LIMITED, Sussex, UK (GB)

(21) Appl. No.: 12/528,288

(22) PCT Filed: Feb. 20, 2008
(86) PCT No.: PCT/GB08/00585

§ 371 Date: Sep. 20, 2010

(30) Foreign Application Priority Data
Feb. 21, 2007 (GB) 0703384.8

(51) Int. Cl. A61C 7/08; A61F 5/56; A61C 5/14

(52) U.S. Cl. 128/848; 433/6; 128/861; 433/24

(57) ABSTRACT

An orthodontic appliance comprising a generally u-shape tooth positioning shell (6,8) adapted for mounting on teeth of the upper or lower arch (2,4) of a patient. The shell (6,8) has a geometry shaped to receive and resiliently apply pressure to one or more teeth to reposition them and at least one additional formation (10,12) on or in the positioning shell adapted to perform a function other than repositioning teeth.

### FIELD OF THE INVENTION

The present invention relates generally to the field of orthodontics and more specifically to orthodontic treatments and appliances that can be used to correct malocclusions (i.e. misalignments of the teeth).

### BACKGROUND

Malocclusion is the misalignment of teeth and/or an incorrect relation between the teeth of the two dental arches, giving rise to faulty contact between upper and lower teeth; i.e. a lack of normal occlusion. In some cases, skeletal disharmony of the face, and in particular an incorrect relationship between the maxilla and mandible, is a contributing factor or even the root cause of malocclusion.

Correcting malocclusions is desirable for functional as well as aesthetic reasons. Uneven distribution of masticatory forces, for example, can result in excessive wear and loosening of teeth and crowding of teeth can mean cleaning is more difficult leading to increased plaque and greater susceptibility to caries.

Conventionally, malocclusions are corrected through the use of orthodontic appliances, commonly known as “braces”, that include a series of metal or ceramic attachments that are cemented to the teeth and connected by tensioned wires that apply forces to the attachments, and hence to the teeth, to give the desired movement. These conventional appliances are, however, not without problems. The procedures for attaching the appliances to the teeth are not straightforward and can cause discomfort, as can the installed appliance. These appliances are generally unsightly and cause problems themselves in maintaining dental hygiene, with the attachments acting as traps for food as it is chewed.

Tooth positioners are an alternative to conventional metal braces that have gained in popularity in recent years.

The concept was first proposed in the 1940s by Dr. Harold Kesling as an appliance for the final positioning of teeth following use of more conventional orthodontic appliances (see: Kesling, “The Philosophy of the Tooth Positioning Appliance”, Am. J. Orthod. Oral. Surg. (1945) 31(6):297-304). Kesling proposed a positioner made of a resilient deformable rubber that had opposed ‘U’-shape channels moulded to fit over the occlusal and incisal surfaces of the upper and lower arches respectively, applying forces to the teeth to influence their position as well as to maintain a desired relationship between the upper and lower arches. Kesling's positioners are also described in his U.S. Pat. No. 2,531,222 and another early example of a tooth positioner is seen in GB 1550777 (Suyehiro).

It is only much more recently, however, that positioners have been proposed as a realistic alternative to conventional metal braces. Recent examples of tooth positioners include those provided by Ortho-Pro-Teknica Ltd under the brand name ClearStep™ and by Align Technology, Inc. under the brand name Invisalign™.

These positioners are moulded polymeric trays or shells of generally U-shape form that fit over the teeth of the upper or lower arch. They are colourless and transparent so are aesthetically much improved compared with the conventional braces. A realignment of the teeth is achieved by using a series of positioners, each positioner typically to be worn for a period of several weeks, to incrementally reposition the teeth. The positioners can be removed by the patient themselves to allow their teeth to be cleaned avoiding the dental hygiene problems associated with the fixings of metal braces.

More severe malocclusions, where there is significant contribution from skeletal disharmony, generally cannot be corrected by re-positioning of teeth alone. Rather, it is necessary to also realign the mandible with the maxilla using other types of functional appliance.

Known tooth borne functional appliances to correct alignment of the mandible and maxilla typically have interacting parts mounted on the teeth of the upper and lower arches that act to posture the mandible forward, bringing about modifications in the alignment of the mandible with the maxilla through stretching of soft tissue and changes in the musculature. One example is a “Twin Block”, a device having maxillary and mandibular portions that may either be removable or cemented to the teeth of the upper and lower arches. The maxillary and mandibular portions have cooperating cam surfaces that engage one another to urge the mandible forwards when the upper and lower arches are brought towards one another.

In cases requiring both realignment of the mandible and repositioning of teeth, a typical approach might be to use a first appliance to broaden the arch, making space for the mandible to be translated forwards using e.g. a twin block appliance and, subsequently using another conventional appliance to finally reposition the teeth.

### SUMMARY OF THE INVENTION

A general aim of aspects of the present invention is to provide methods and appliances that can correct malocclusions, especially but not necessarily exclusively those that result at least in part from skeletal disharmony of the face, preferably in a manner that is more straightforward than the known approaches discussed above.

In particular, aspects of the present invention propose a combined tooth positioner and functional appliance that can be used to simultaneously reposition teeth (e.g. in the manner of a conventional positioner) and provide an additional orthodontic and/or orthopaedic function (e.g. forward posturing of the mandible to correct skeletal disharmony), as well as methods of combined treatment including the repositioning of teeth simultaneously with another functional orthodontic and/or orthopaedic treatment (e.g. a dentofacial orthopaedic treatment).

Thus, some embodiments of the invention provide functional appliances adapted for orthopaedic correction of jaws, providing simultaneous orthopaedic correction and alignment of the teeth. Functional appliances in accordance with embodiments can be used, in particular, in the growing child to optimise facial growth, in particular jaw growth.

In preferred aspects, a series of combined positioner/functional appliance devices are provided for sequential use to incrementally reposition teeth and treat another orthodontic condition (e.g. skeletal disharmony).

In a first aspect there is provided an orthodontic appliance comprising:
a generally u-shape tooth positioning shell adapted for mounting on teeth of the upper or lower arch of a patient, the shell having a geometry shaped to receive and resiliently apply pressure to one or more teeth to reposition them; and
at least one additional formation on or in the positioning shell adapted to perform a function other than repositioning teeth.

The appliance may further comprise a second generally u-shape tooth positioning shell adapted for mounting on teeth of the other of the upper and lower arch of the patient, the second positioning shell having a geometry shaped to receive and resiliently apply pressure to one or more teeth to reposition them.

The second positioning shell may also comprise at least one additional formation on or in the positioning shelf adapted to perform a function other than repositioning teeth.

The additional formations of the two positioning shells may cooperate with one another to provide the desired function.

Examples of functions that the additional formation(s) may perform include orthopaedic functions, especially dentofacial orthopaedic functions such as: posturing of the mandible to correct skeletal disharmony of the face (e.g. to serve in the manner of a twin-block or Bionator appliance); posturing of the mandible to help prevent snoring; and controlled correction of patients with Temporomandibular Joint Disfunction Syndrome (‘TMD Syndrome’) by posturing the jaw to permit decompression of the temporomandibular joint and allow relaxation and reestablishment of the muscles and ligaments surrounding the joint, eventually leading to reestablishment of the normal architecture and form of the affected temporomandibular joint

Particularly when adapted from the treatment of TMD Syndrome, the appliance may be adapted to permit super eruption of the posterior teeth in mandible and/or maxilla to help re-establish the vertical dimension.

Another example of a function that might be provided in addition to alignment of teeth is the provision of cushioning to protect the teeth from impact (e.g. to serve as a gum shield for sport).

In some embodiments, one of more of the additional formations are protrusions. They may for example be external protrusions on the outer surface of the shell, e.g. on the occlusal surface of the shell. Additionally or alternatively there may be protrusions or thickenings within the shell.

One or more of the additional formations may create superficial bite planes or guidance planes (e.g. anterior or posterior bite planes) to disclude the occlusion, e.g. to permit correction of crossbites or to help in the controlled eruption of developing permanent teeth in the growing and developing child.

In some embodiments, one or more of the additional formations may be cavities or apertures in the positioning shell. For instance, one or more channels may be formed to help control eruption of permanent teeth in the growing and developing child.

The additional formations may comprise cushioning, for example cushioning between an external surface of the formation and an inner surface of the positioning shell to cushion one or more teeth against external forces applied to the formation.

A series of appliances in accordance with the first aspect, each successive appliance having a geometry that differs in one or more respects from the preceding appliance in the series, can be used to incrementally treat a patient, the patient wearing the appliances of the series sequentially, in turn, each for a period of time.

Accordingly, in another aspect there is provided a series of orthodontic appliances according to the first aspect, each successive appliance having a different geometry to the preceding appliance in the series.

The differences in geometry may be differences in the geometry of the shell shaped to receive and resiliently apply pressure to one or more teeth to reposition them and/or the geometry of the additional formation(s).

The series of appliances may be provided to a patient together in a single package.

In a second aspect there is provided an orthodontic appliance comprising:
an upper generally u-shape shell adapted for mounting on teeth of the upper arch of a patient;
a lower generally u-shape shell adapted for mounting on teeth of the lower arch of the patient; and
additional formations protruding from the occlusal surfaces of the upper and lower shells, the protruding formations cooperating with one another in use to urge the mandible in a predefined direction.

The additional formations may cooperate to urge the mandible forward with respect to the maxilla.

The additional formations may have cam surfaces that engage one another in use, e.g. as the jaw is closed, to urge the mandible in the predefined direction (e.g. forward with respect to the maxilla).

A series of appliances in accordance with the second aspect, each successive appliance having a geometry that differs in one or more respects from the preceding appliance in the series, can be used to incrementally reposition the patient's mandible, the patient wearing the appliances of the series sequentially, in turn, each for a period of time.

Accordingly, in another aspect there is provided a series of orthodontic appliances according to the second aspect, the additional formations of each successive appliance having a different geometry to those of the preceding appliance in the series.

The series of appliances may be provided to a patient together in a single package.

In a third aspect there is provided a method of orthodontic treatment comprising using an orthodontic appliance to simultaneously reposition one or more teeth and to provide another orthodontic function, the orthodontic appliance comprising:
a generally u-shape tooth positioning shell mounted on teeth of the upper or lower arch of a patient, the shell having a geometry shaped to receive and resiliently apply pressure to one or more teeth to reposition them; and
at least one additional formation on or in the positioning shell adapted to perform the other orthodontic function.

Examples of orthodontic functions that may be provided in combination with tooth positioning in accordance with this aspect include: posturing of the mandible to correct skeletal disharmony of the face (e.g. to serve in the manner of a twin-block or Bionator appliance); posturing of the mandible to help prevent snoring; and provision of cushioning to protect the teeth from impact (e.g. to serve as a gum shield for sport).

In some embodiments, the combined orthodontic treatment will be carried out using a plurality of orthodontic appliances, each having the form set out above, that are worn sequentially by the patient over a period of time to treat the patient in increments, i.e. incrementally repositioning the teeth and/or incrementally bringing about other changes in the facial skeleton, e.g. to correct skeletal disharmony.

Each successively used appliance may have a geometry that differs incrementally from the previous appliance to bring about the desired incremental changes. The differences in geometry may be differences in the geometry of the shell shaped to receive and resiliently apply pressure to one or more teeth to reposition them and/or the geometry of the additional formation(s).

In a fourth aspect the invention provides a method of treating a skeletal disharmony of a patients face, the method comprising using a series of orthodontic appliances according the second aspect above to incrementally reposition the patient's mandible relative to their maxilla, the additional formations of each successive appliance having a different geometry to those of the preceding appliance in the series.

### BRIEF DESCRIPTION OF DRAWINGS

Embodiments of the invention will now be described, by way of example only, with reference to the accompanying drawings, in which:

FIG. 1 shows a side view of the upper and lower arches of a patient with a combined tooth positioner and twin block appliance in accordance with an embodiment of the invention installed;

FIGS. 2a to 2d illustrate (in side view) the use of a series of combined tooth positioner and twin block appliances to incrementally translate a patient's mandible forwards and simultaneously reposition one or more teeth;

FIGS. 3a and 3b show plan views of the lower and upper arches of the patient with the combined appliance illustrated in FIG. 1a applied (i.e. at the beginning of a treatment); and

FIGS. 4a and 4b show plan views of the lower and upper arches of the patient with the combined appliance illustrated in FIG. 1d applied (i.e. after a series of incremental movements).

### DESCRIPTION OF EMBODIMENT

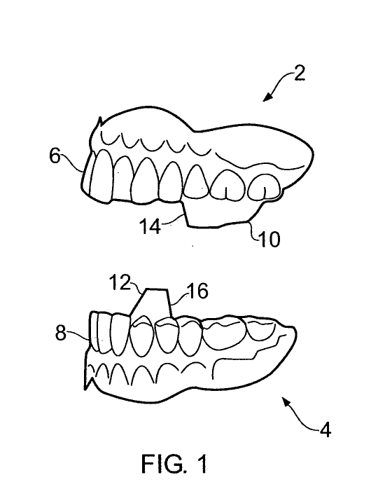

FIGS. 1 to 4 illustrate orthodontic appliances that can be used in accordance with an embodiment of the present invention to simultaneously reposition teeth and reposition a patient's mandible with respect to their maxilla in an incremental fashion. Conveniently, this approach enables the functions of a conventional tooth positioner and a conventional twin-block appliance to be combined in a single appliance, potentially giving benefits including the reduction in both the complexity of a treatment and the time for a treatment to be executed. Other embodiments have the additional advantage that they can provide a very effective and convenient approach to the treatment of TMD syndrome, helping to re-establish normal function in the muscles and ligaments on the tempromandibular joint of patients with this debilitating condition.

FIG. 1 shows a two part orthodontic appliance in situ on the upper and lower arches 2, 4 of a patient (the arches having been drawn separated from one another for ease of illustration). The appliance comprises upper and lower shells 6, 8 that are generally U-shape to fit over the patient's teeth. In this example the shells are formed from a polymeric, preferably transparent material. Suitable materials include PET.

The geometry of each shell can be designed, as with known ‘positioners’, to receive and resiliently apply pressure to one or more teeth in order to reposition them.

In contrast to known positioners, however, as seen in FIG. 1, each of the upper and lower shells 6, 8 has a pair of protuberances 10, 12 on its occlusal surface (only one of each pair is seen in FIG. 1, the other of the pair being symmetrically positioned on the other side of the respective arch). The protuberances 10, 12 are preferably formed integrally with the shells 6, 8, as seen in this example.

In the illustrated example the upper protuberances 10 are positioned below the patient's maxillary molars and the lower protuberances 12 are positioned above the patient's mandibular premolars. Alternative positions are possible depending on the intended treatment.

Each of the protuberances 10, 12 is formed with a cam surface 14, 16. In the case of the upper protuberances 10 the cam surface 14 is the foremost surface, whereas it is the rearmost surface that serves as the cam surface 16 for the lower protuberances 12. In use, when the patient closes their jaw, the cam surfaces 14, 16 of respective pairs of upper and lower protuberances 10, 12 on each side of the mouth engage one another and act to posture the patient's mandible forwards with respect to the maxilla.

Each of the protuberances 10, 12 can be formed as a solid block of material (e.g. the same polymeric material as the rest of the appliance). Alternatively, the protuberances 10, 12 may be hollow, the wall thickness of the protuberance being selected to give the desired stiffness to function. If desired, cushioning (not shown) can be incorporated in the upper and/or lower protuberances 10, 12 to minimise the forces applied to the teeth as the jaw is postured forwards. For instance, the protuberances 10, 12 can be filled (at least partly but more preferably completely) with a cushioning material, e.g. a silicone material.

The illustrated appliance, comprising upper and lower shells 6, 8, can therefore act to apply forces to reposition one or more teeth and at the same time to posture the mandible forwards, simultaneously performing the functions previously performed separately using positioners (or other teeth aligning appliances) and twin-block appliances applied to the teeth in separate, successive, treatment steps. The shells 6, 8 can, for instance, be vacuum formed over a mould based on the patient's upper or lower arch 2, 4 (as the case may be), in a similar fashion to the methods used for manufacturing known positioners.

In practice a series of these polymeric shells 6, 8 is manufactured and used to incrementally reposition one or more teeth from an initial configuration to a final configuration via one or more intermediate configurations. At the same time, the jaw (mandible) can be incrementally repositioned from a starting position (relative to the maxilla) to a final desired position.

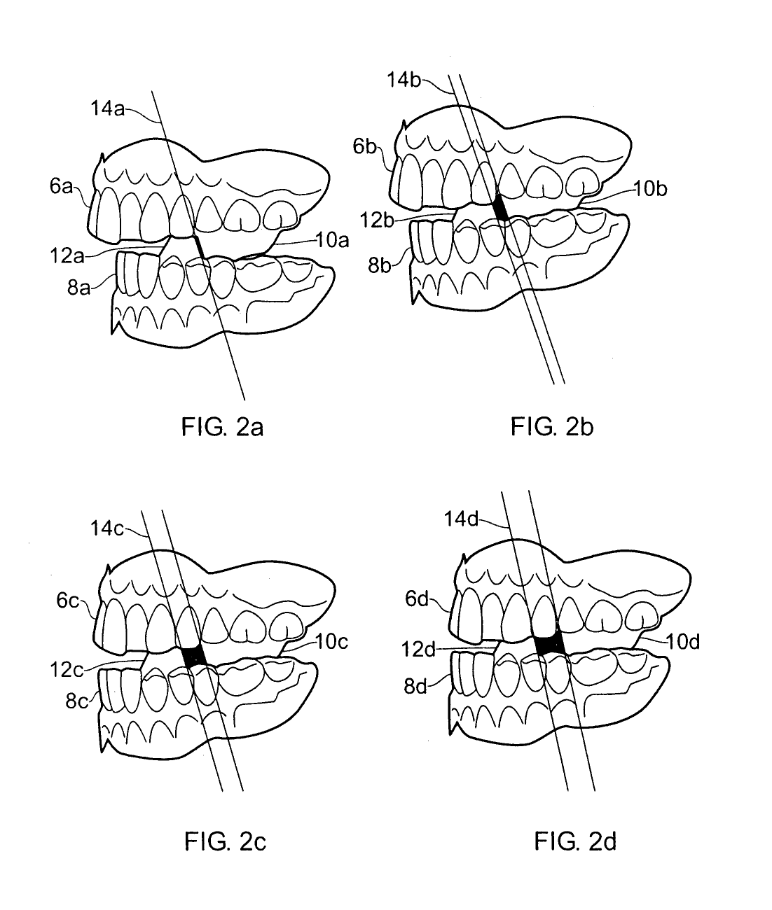

At each increment, as illustrated in FIGS. 2a to 2d (showing 4, sequential incremental steps in a treatment), the relative geometry of the protuberances 10a-d, 12a-d can be adjusted to posture the jaw a little further forward, until the final desired position is reached. In this example, the lower protuberances 12a-d are in fact not modified. The cam surface 14a-d of the upper protuberances 10a-d, however, is extended forward at each increment. In FIG. 2d, for example, it can be seen that the cam surface 14d is as far forward as the second premolar, compared with its position in line with the first molar in FIG. 2a.

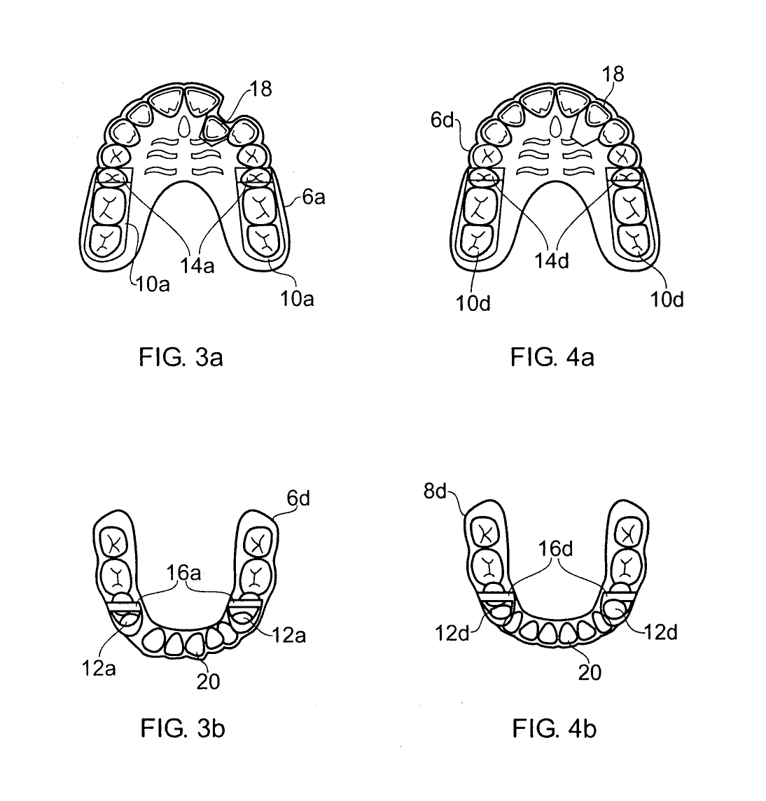

At the same time, the geometry of the generally U-shaped channel in each subsequent shell 6, 8 in the series can, if necessary, also be changed in order to incrementally reposition one or more teeth, as with conventional positioners. For instance, in this example, looking at FIGS. 3 and 4, it can be seen that the appliance is used to translate the upper left lateral incisor 18 outwardly into correct alignment with the rest of the upper arch 2 and to rotate the lower left central incisor 20 into correct alignment. FIGS. 3 and 4 show the arches 2, 4 pre-treatment and at the end of the treatment. The teeth would be repositioned in a series of steps between these two extremes.

The shells 6, 8 of the appliance will typically be manufactured and issued to a patient in sets. A set of shells will typically include three, four, five, six, seven, eight or more pairs (upper and lower) of shells provided as a batch to the patient. Each pair of shells 6, 8 will typically be worn for several weeks.

In some cases, one set of shells will be sufficient to complete a treatment. In other cases, multiple sets of shells will be needed. In the latter case, new impression will generally be taken from the patient between sets in order that the next set can be based closely on the actual configuration of the patient's teeth mid-treatment.

1. An orthodontic appliance comprising:
a generally u-shape tooth positioning shell adapted for mounting on teeth of an upper or lower arch of a patient, the shell having a geometry shaped to receive and resiliently apply pressure to one or more teeth to reposition them; and
at least one additional formation on or in the positioning shell adapted to perform a function other than repositioning teeth.

2. An orthodontic appliance according to claim 1, further comprising a second generally u-shape tooth positioning shell adapted for mounting on teeth of the other of the upper and lower arch of the patient, the second positioning shell having a geometry shaped to receive and resiliently apply pressure to one or more teeth to reposition them.

3. An orthodontic appliance according to claim 2, wherein the second positioning shell comprises at least one additional formation on or in the positioning shell adapted to perform a function other than repositioning teeth.

4. An orthodontic appliance according to claim 3, wherein the additional formations of the two positioning shells cooperate with one another to provide the desired function.

5. An orthodontic appliance according to claim 1, wherein the function that the additional formation perform include one or more functions selected from: orthopaedic functions; posturing of the mandible to help prevent snoring; controlled correction of patients with Temporomandibular Joint Disfunction Syndrome; and cushioning to protect the teeth from impact.

6. An orthodontic appliance according to claim 5, wherein the orthopaedic functions are dentofacial orthopaedic functions such as posturing of a mandible to correct skeletal disharmony of a face.

7. An orthodontic appliance according to claim 1, wherein the additional formation is a protrusion.

8. An orthodontic appliance according to claim 7, wherein the protrusion is an external protrusion on the outer surface of the shell.

9. An orthodontic appliance according to claim 8, wherein protrusion is on an occlusal surface of the shell.

10. An orthodontic appliance according to claim 1, wherein the additional formation comprises a protrusion or thickening within the shell.

11. An orthodontic appliance according to claim 1, wherein the additional formations create superficial bite planes to permit correction of crossbites or to help control eruption of developing permanent teeth in a child.

12. An orthodontic appliance according to claim 1, wherein the additional formation is a cavity or aperture in the positioning shell.

13. An orthodontic appliance according to claim 12, wherein the cavity or aperture is a channel to help control eruption of permanent teeth in a child.

14. An orthodontic appliance according to claim 1, wherein the additional formation comprises cushioning.

15. An orthodontic appliance according to claim 14, wherein the cushioning is between an external surface of the formation and an inner surface of the positioning shell to cushion one or more teeth against external forces applied to the formation.

16. A series of orthodontic appliances, each appliance being an appliance according to claim 1, each successive appliance having a different geometry to the preceding appliance in the series.

17. A series of orthodontic appliances according to claim 16, wherein the differences in geometry comprise differences in the geometry of the shell shaped to receive and resiliently apply pressure to one or more teeth to reposition them.

18. A series of orthodontic appliances according to claim 16, wherein the differences in geometry comprise differences in the geometry of the additional formation.

19. A series of orthodontic appliances according to claim 1 provided together in a single package.

20. An orthodontic appliance comprising:
an upper generally u-shape shell adapted for mounting on teeth of the upper arch of a patient;
a lower generally u-shape shell adapted for mounting on teeth of the lower arch of the patient; and
additional formations protruding from an occlusal surfaces of the upper and lower shells, the protruding formations cooperating with one another in use to urge the mandible in a predefined direction.

21. An orthodontic appliance according claim 20, wherein the additional formations cooperate to urge the mandible forward with respect to the maxilla.

22. An orthodontic appliance according claim 20, wherein the additional formations have cam surfaces that engage one another in use, e.g. as the jaw is closed, to urge the mandible in the predefined direction.

23. A series of orthodontic appliances, each appliance being an appliance according to claim 20, the additional formations of each successive appliance having a different geometry to those of the preceding appliance in the series.

24. A series of orthodontic appliances according to claim 23 provided together in a single package.

25. A method of orthodontic treatment comprising;
using an orthodontic appliance to reposition one or more teeth, the orthodontic appliance including a generally u-shape tooth positioning shell mounted on teeth of the upper or lower arch of a patient, and at least one additional formation on or in the positioning shell adapted to perform another orthodontic function, the shell having a geometry shaped to receive and resiliently apply pressure to and reposition one or more teeth; and
simultaneously using the orthodontic appliance to provide said another orthodontic function.

26. The method of claim 23, further comprising using a series of orthodontic appliances for treating a skeletal disharmony of a patients face, the patient wearing the appliances of the series in succession to incrementally reposition the patient's mandible relative to the patient's maxilla.
